(19) United States
(12) Patent Application Publication
(10) Pub. No.: US 2013/0280291 A1
(43) Pub. Date: Oct. 24, 2013

(54) Mineral Pitch Resin manufactured under a safe and low temperature procedure.

(71) Applicant: Nodari Rizun, Los Angeles, CA (US)

(72) Inventor: Nodari Rizun, Los Angeles, CA (US)

(21) Appl. No.: 13/856,440

(22) Filed: Apr. 4, 2013

Related U.S. Application Data
(60) Provisional application No. 61/636,008, filed on Apr. 20, 2012.

(51) Int. Cl. A61K 35/02

(52) U.S. Cl. CPC A61K 35/02
USPC 424/195.18; 530/200

(57) ABSTRACT

The invention claims a manufacturing method of the Mineral Pitch Resin also known as shilajit, silajit, mumije, mumie, momie, moomie, mumio, momia, brashgun, etc., and product variations thereof. The method produces genuine Mineral Pitch Resin, under a predetermined controlled method. Such method results into the Resin, which is verifiably free from harmful contaminants and is safe for humans to consume as a healing, tonifying and adaptogenic substance. Such resin passes authenticity tests, is of consistent quality and has preserved heat sensitive biologically active substances innate to raw material used to manufacture the Mineral Pitch.

### BACKGROUND OF THE INVENTION

This inventions pertains to manufacturing of a premium and genuine mineral pitch resin (also known as shilajit, silajit, salajeet, mumije, mumie, momie, moomie,mumio, momia, brashgun, etc.) and formulations made with such resin, which itself is a potent medicinal, adaptogenic, nutraceutical, cosmetic and nutritional substance.

Mineral Pitch, known by multiple names in different cultures (shilajit, silajit, salajeet, mumije,mumie, momie, moomie,mumio, momia, brashgun, etc.) is one of the most potent adaptogenic stimulators known to mankind. It has been successfully used for thousands of years to by humans and animals to treat diseases, injuries and address nutritional deficiencies. Modern medical and nutritional science has confirmed and identified multiple health related benefits of the Resin.

The most common, but not only, known origin of Mineral Pitch is located in the mountains of various regions in Asia, Caucasus, Europe, Antarctica, etc. Traditionally, prior to advent of advanced pharmaceutical and chemical science, Mineral Pitch was primitively processed by intense methods of purification and heating.

The problems are that, both undue purification and/or excessive heating take the Mineral Pitch out of its traditional environment and often render it somewhat unsafe or less efficient. Purification can cause Mineral Pitch to be contaminated with harmful impurities. Excessive heating, usually over 39 degrees Celsius, will destroy multiple, valuable compounds such as enzymes, vitamins, and microflora indigenous to the Mineral Pitch.

Another substantial problem is counterfeit Shilajit imitation products by multiple manufacturers. Such imitation products usually come as powders and don't have most benefits of genuine Mineral Pitch Resins.

Popularity of the holistic, superfood and natural medicinal substances makes consistent low temperature and non-toxic, non-harsh purification process in the manufacturing of Mineral Pitch highly relevant. Absence of a standardized and precisely controlled manufacturing method for sourcing, collection, transportation, purification, concentration and packaging of Mineral Pitch is limiting for mass manufacturing of a safe, potent and sufficient market supply of a “live” Mineral Pitch resin paste.

This patent application solves the challenge. The novelty is stipulated by manufacturing Mineral Pitch resin in a production form and way which is consistently genuine, pure, safe, and temperature-controlled throughout the manufacturing cycle. This renders the Mineral Pitch to be qualified as “raw-live” and of a supreme quality. The invention also overcomes the challenge of safe and consistent mass manufacturing of this live nutriceutical. The product is a resin/paste form and not powder.

### BRIEF SUMMARY/DESCRIPTION OF THE INVENTION

The invention deals with full cycle of manufacturing the premium quality Mineral Pitch Resin and formulations made with it in a way which results in superior quality and safety from microbiological, mineral and radiological contaminants in a controlled environment.

The Invention covers all the stages of the sourcing, logistics and manufacturing process of genuine Mineral Pitch Resin (also known as shilajit, silajit, mumije,mumie, momie, moomie,mumio, momia, brashgun, etc.) as well as manufacturing of said Resin with botanicals and various medicinal or cosmetic ingredients mixed into it. In contrast to traditional methods it implies a controlled and standardized procedure, which results resin of superior quality, safe for end consumers with intact heat sensitive enzymes and biologically active substances. In contrast to multiple imitation products the invention produces the Resin, which is safe to consume and has an effect closest to the pure raw material in nature with the innate biologically active substances. The said resin is not treated with hot organic solvents, such as ethanol or methanol, is not artificially oxygenated or acidified to boost any of the fractions of the resin's fractions. The resulting product, which is not a powder, with substantial glycine content, which is not fortified with fulvic acid or any other fillers and naturally passes authenticity tests for mineral pitch resins.

### DETAILED DESCRIPTION OF THE INVENTION

Safe temperature regimes and grades consistently used: under 39 C (102.2 Fahrenheit)—1^{st} grade premium quality, under 48 C (118.4 Fahrenheit)—2^{nd} grade quality, higher temperatures—3^{rd} grade quality.

Major tools and utensils: transportation containers supplied with UV light and temperature control or as alternative temperature controlled containers, clean rooms, clean room dehumidifiers, sterile vessels equipped with mixers and temperature control with or without dehumidifiers/demoisturizers, UV lamps or other source of light, sound or electro-magnetic disinfection, cutting—shaving—grating utensils, opaque transportation containers, biophotonic glass or apothecary jars, food grade alcohol or other food grade disinfectant.

Stages: 1. preparation 2. identification 3. collection 4. transportation 5. purification 6. concentration/demoisturization 7. packaging 8. transportation 9. repackaging

### Methodology

All stages, with the partial exception of (8) transportation, take place at a constantly monitored and maintained environment, within a consistent temperature range of under 39 degrees Celsius for the first grade quality of product during the stages of manufacturing and under 48 degrees Celsius for the second grade quality of product and higher temperatures for the third grade quality.

Upon initiating the preparation cycle, the manufacturer is required to prepare the clean room facility, equipped with UV sterilization light (or other source of light, sound or any other form of electro-magnetic disinfection), dehumidifiers, temperature controlled increased area mixing or still vessels (alternatively equipped with dehumidifiers/demoisturizers), purified or sterilized (treated) water or liquid herbal extraction, transportation containers equipped with features of UV or any other form of disinfection or any other temperature control. Once the preparation cycle is complete, one may proceed to identification and collection of the raw material.

Once the raw material is visually and/or chemically identified as the desired Mineral Pitch, a person collecting the material cleans (cuts, grates or shaves off) off the external layers of the identified Mineral Pitch. Next the collected material is briefly washed off with food grade alcohol and immediately placed in the thermo-controlled containers with or without UV sterilization light (or any other source of sterilization) activated. An alternative would be a container without a sterilization light (with or without any other source of sterilization). Raw materials from different types of containers are not combined until a laboratory analysis confirms processed materials for safety and uniformity. At the stage of collection the natural (weather) and controlled environment are to be monitored to stay under 39C or 48C. If the temperature exceeds 39C the raw materials for the 1^{st} quality grade are not collected, if the temperature exceeds 48C the raw materials for the second quality grade are not collected. Any raw material collected under ambient temperatures of over 48C are to be placed n batches for the 3^{rd} degree quality.

Once collected into the temperature controlled containers equipped with a source of disinfection, or alternatively without one, the raw material is transported into the clean room facility for processing. Once delivered into the facility all operations take place in the clean rooms under exposure to UV light (or any other mentioned above source of disinfection). Alternatively the process can take place in the clean rooms without a UV light source of disinfection. The raw initially cleaned material is taken out from the containers and is once again very briefly washed/wiped with food grade alcohol. Next the material of different temperature grades is separated and dissolved in sterile or other form of water or liquid (deionized, purified, mineralized, geographically sourced, magnetically treated, structured, informationally programmed, etc.). The solution is filtered multiple times through filters in order to rid off impurities, sediment, sand, etc. The process of adding water and filtration may be repeated up to 50 times with various types of filters, ensuring elimination of particles as small as several micrometers or several hundred nanometers. Once the filtration is complete the filtrate may or may not be mixed with other desired herbal extracts, minerals or other substances which would be beneficial to the efficacy of the final product/Mineral Pitch formulation. All of the procedures mentioned in this paragraph take place within temperature-controlled environments of under 39C for the 1^{st} grade quality and under 48 degrees C. for the second grade quality. This process may take place in vacuum, under pressure or exposure to different sound or electromagnetic waves.

Next the filtrate is transferred to the temperature controlled vessel equipped, or not, with an inbuilt dehumidification system and a mixer or stirrer. The alternative to the inbuilt dehumidification/demoisturization system can be a dehumidification system as a part of the clean room environment. The filtrate is mixed and dehumidified/demoisturized, which turns it into a resin/paste form. Generally the resin will have a humidity level under but not limited to 20%. All of the procedures mentioned in this paragraph take place within a clean room, temperature controlled environment of under 39C for the 1^{st} grade quality and under 48 degrees C. for the second grade quality and above for the third grade quality.

Each manufactured batch is laboratory tested for: purity, secondary authenticity, heavy metal contamination, radiation contamination, and microbiological contamination. If any of these tests fail, the batch is immediately discarded. If the batch passes the tests, the product is ready for packaging. The resin is packaged in a clean room environment, with an active source of disinfection as mentioned prior, on or off. The resin is then transferred into transportation containers, which isolate the resin from light and external environment. All of the procedures mentioned in this paragraph take place within a clean room, temperature controlled environment of under 39C for the 1^{st} grade quality and under 48C degrees for the second grade quality and above for the third grade. At this point the resin is preserved and is preserved in the “hibernation” mode. The containers are moved to the repackaging facility. Maintenance of strict temperature regimes, while in “hibernation” mode is not required.

The manufacturer may or may not maintain a strict temperature regime of under 39 degrees Celsius, while transporting the Mineral Pitch for repackaging.

Next, repackaging takes place. During the repackaging the resin is transferred into individual, high quality biophotonic or apothecary glass. All of the procedures mentioned in this paragraph take place within temperature regimes of under 39C for the 1^{st} grade quality and under 48 degrees C. for the second and higher temperatures for the third grade quality. From this point on, the resin is protected from external environment and can be used for final consumption.

This application claims the following:

1. A method of manufacturing a mineral pitch product covering the sourcing, purification, adding botanicals or substances, demoisturization and packaging under a controlled environment resulting a biologically and or enzymatically active product which is either a resin or a solid or a semisolid or a liquid or a paste.
1.2 Where from the moment of identification in nature or at the source the resin is transported in hygienic and temperature controlled containers capable of maintaining the internal temperature at or under 39 degrees Celsius for the 1^{st} grade quality and a predetermined temperature for other quality grades.
1.1 Where the initial treatment of the material takes place in a clean room environment of class D and higher or class ISO 8 and higher or any classes with identical, analogue or similar characteristics.
1.2. Where predetermined spectrums of light, sound or electro-magnetic rays are used for disinfection, purification, encoding or structuring at any stage of the manufacturing process.
1.3. Where the manufacturing process consists fully or partially of such stages as site preparation, raw material identification, raw material collection, raw material transportation, purification, dehumidification or demoisturization, adding substances, packaging for transportation, packaging for storage or final packaging.
1.4. Where the full manufacturing or any one of the individual manufacturing or packaging stages of the cycle(i.e. extraction from the transportation containers, initial purification, mixing with botanicals or other substances, dehumidification or demoisturturization and packaging) takes place in a clean room environment of class D and higher or class ISO 8 and higher or any classes with identical or similar characteristics.
1.5. Where the full manufacturing cycle or parts thereof take place under predetermined temperature regimes.
1.6. Where the full manufacturing cycle or parts thereof take place under temperatures of less or equal to 39C or less or equal to 48C or less or equal 60C or other temperature regimes.
1.7. Where the raw material is initially dissolved (purified) in liquid which can be predetermined type of water or an alternative to water.
1.8. Where said water or an alternative can be of is either drinking or purified or highly purified or injectable or deionized or distilled or disinfected, or other type or grade.
1.9. Where in addition such water or liquid can be mineralized and or geographically sourced and or magnetically treated and or structured and or informationally programmed or encoded.
1.10. Where the resulting solution or parts thereof are filtered once or multiple times through various filters ensuring elimination of particles ranging in size from several centimeters to millimeters several micrometers and several hundred nanometers. Such filtration can take under pressure or vacuum or other physically or environmentally controlled conditions if necessary.
1.11. Where botanicals or their extracts or any substances beneficial to human or animal health may be added to or mixed with the materials at any stage while it is still in a liquid or a semi-liquid state.
1.12. Where the filtrate with or without botanicals added to it is dehumidified/demoisturized in order to get rid of excess moisture and result into a paste or resin.
1.13. Where the vessels used for dehumidification/demoisturerization are mixing the said liquid and or equipped with temperature control.
1.14. Where the said process can take place under vacuum or pressure or under a predetermined light spectrum or sound wavelength.
1.15. Where the resulting product is tested for purity, primary and secondary authenticity, heavy metal contamination, radiation contamination, microbiological contamination. Whereby if any of the said tests fail the full manufactured batch is discarded.
1.16. Where the resulting product is a resin or paste or solid or semi-solid or liquid and not a powder and can be identified as a genuine resin.
1.17. Where the said material is packaged either into transportation or biophotonic glass containers.
1.18. Where opaque or biophotonic glass containers are used to storage and preserve the resin.

2. Mineral Pitch Resin also known as shilajit or silajit or mumije or mumie or momie or moomie or mumio or momia or brashgun or alternatively any other analogue product, which is sourced and manufactured consistently under a predetermined controlled procedure resulting into a resin or paste or solid or semi-solid or liquid.

3. Mineral Pitch product also known as shilajit, silajit, salajeet, mumije, mumie, momie, moomie, mumio, momia, brashgun, etc., in a solid, semi-solid state which is safe for human consumption with identifiable biologically heat sensitive active substances which could be found in raw materials used to manufacture said product.
